(19) United States
(12) Patent Application Publication
(10) Pub. No.: US 2021/0301248 A1
(43) Pub. Date: Sep. 30, 2021

(54) EVALUATOR AND EVALUATION METHOD FOR EVALUATING DIFFERENTIATION LEVEL OF CULTURED CELLS, AND AUTOMATIC CELL CULTURE SYSTEM

(71) Applicant: Hitachi, Ltd., Tokyo (JP)

(72) Inventors: Hikaru SAITO, Tokyo (JP); Midori KATO, Tokyo (JP); Kakuro HIRAI, Tokyo (JP); Masaharu KIYAMA, Tokyo (JP); Kunio OHYAMA, Tokyo (JP); Hiroko HANZAWA, Tokyo (JP); Shizu TAKEDA, Tokyo (JP)

(73) Assignee: HITACHI, LTD.

(21) Appl. No.: 16/951,244

(22) Filed: Nov. 18, 2020

(30) Foreign Application Priority Data
Mar. 27, 2020 (JP) 2020-057785

(51) Int. Cl. C12M 1/36; C12M 1/34; G01N 35/00; G01N 21/64

(52) U.S. Cl. CPC C12M 41/48; C12M 41/32; G01N 2021/6439; G01N 21/645; G01N 21/6428; G01N 35/00594

(57) ABSTRACT

Provided are an evaluator including an analysis unit that evaluates a differentiation level of a cultured cell on the basis of a content of a component in a culture supernatant of the cultured cell, and an automatic cell culture system including the evaluator and an automatic culture device.

### CROSS-REFERENCE TO RELATED APPLICATIONS

The present application claims priority to Japanese Patent Application No. 2020-057785 filed on Mar. 27, 2020 which is incorporated herein by reference.

### BACKGROUND OF THE INVENTION

### 1. Field of the Invention

The present invention relates to an evaluator and an evaluation method for evaluating a differentiation level of cultured cells, and an automatic cell culture system.

### 2. Description of the Related Art

In order to stabilize and improve quality of cell production using an automatic cell culture device, it is important to monitor a culture state of cells from an outside and perform control in accordance with the culture state without taking out the cells.

In JP 2019-154283 A, it is disclosed that exosomes were recovered from osteoblasts obtained by inducing differentiation, and when binding ability to 45 kinds of lectins was evaluated, a plurality of kinds of sugar chains that are more frequently expressed on surfaces of exosomes derived from differentiation-induced osteoblasts were successfully found.

### SUMMARY OF THE INVENTION

An object of the present invention is to provide an evaluator and an evaluation method for evaluating a differentiation level of a cultured cell, and an automatic cell culture system.

One aspect of the present invention is an evaluator for evaluating a differentiation level of a cultured cell, and the evaluator includes an analysis unit that evaluates the differentiation level of the cultured cell on a basis of a content of a component in a culture supernatant of the cultured cell.

Another aspect of the present invention is an automatic cell culture system including: a culture device including a cell culture container for culturing a cell, and a drainage container for discarding a culture supernatant used for cell culture in a predetermined period; and an evaluator including a measurement unit for measuring a content of a component in the culture supernatant; and an analysis unit that evaluates a differentiation level of the cell from the content.

A further aspect of the present invention is an evaluation method for evaluating a differentiation level in a cultured cell, the method including evaluating a differentiation level of a cultured cell on a basis of a content of a component in a culture supernatant of the cultured cell.

The present invention has made it possible to provide an evaluator and an evaluation method for evaluating a differentiation level of a cultured cell, and an automatic cell culture system. Problems, configurations, and effects of the present invention other than those described above will be clarified by description of modes for carrying out the invention described below.

### BRIEF DESCRIPTION OF THE DRAWINGS

FIG. 1 is a schematic diagram showing a configuration of an evaluator that is one embodiment of the present invention;

FIG. 2 is a schematic diagram showing a configuration of an automatic cell culture system that is one embodiment of the present invention;

FIG. 3 is a flowchart of a control method for an automatic cell culture system according to one embodiment of the present invention;

FIG. 4 is a graph showing change in exosome density per 1 mL of culture supernatant in a differentiation induction process from iPS cells to dopamine neural progenitor cells in one example of the present invention;

FIG. 5 is a graph showing change in a content of CD63 per 1 mL of culture supernatant in a differentiation induction process from iPS cells to dopamine neural progenitor cells in one example of the present invention;

FIG. 6 is graphs showing change in contents of CD63, CD81, CD9, TSPAN9, HSP70, and HSP90 per 1 mL of culture supernatant in a differentiation induction process from iPS cells to dopamine neural progenitor cells in one example of the present invention;

FIG. 7 is a graph showing change with time in a content of CD63 per amount of culture supernatant after medium replacement in the differentiation induction process from iPS cells to dopamine neural progenitor cells in one example of the present invention; and

FIG. 8 is a graph showing correlation analysis between a content of CD63 on the 8th day and an expression amount of markers of dopamine neural progenitor cells on the 12th day in one example of the present invention.

### DESCRIPTION OF THE PREFERRED EMBODIMENTS

Various preferred embodiments of the present invention will be described below with reference to the drawings and examples. However, these preferred embodiments are merely examples for realizing the present invention, and do not limit the technical scope of the present invention. Note that in each figure, the same reference numerals are given to common configurations.

### ==Evaluator for Evaluating a Differentiation Level of Cultured Cells==

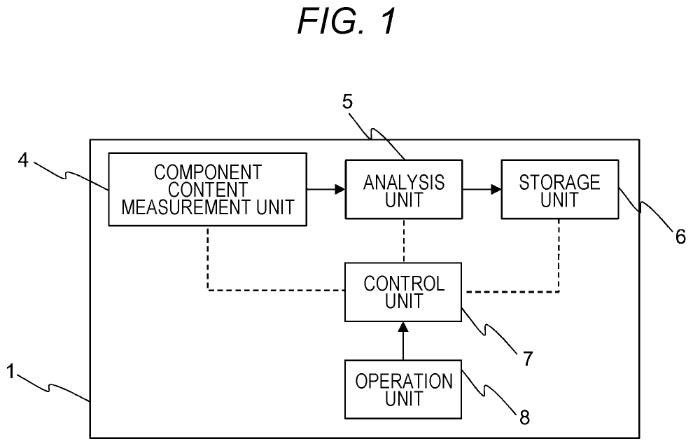

An evaluator for evaluating a differentiation level of cultured cells disclosed in the present specification includes an analysis unit that evaluates the differentiation level of the cultured cells on the basis of a content of a component in a culture supernatant of the cultured cells. Hereinafter, the evaluator of the present embodiment will be described in detail with reference to FIG. 1. Note that in the present specification, the culture supernatant refers to a medium for culture used for culture for a predetermined time. The medium for culture at the start of culture is also included in the culture supernatant of the present disclosure.

An evaluator 1 shown in FIG. 1 includes a measurement unit 4 for measuring the content of components in the culture supernatant, an analysis unit 5 for evaluating the differentiation level of the cultured cells on the basis of the content, a storage unit 6 that stores data obtained by the analysis, a control unit 7 that controls the measurement unit 4, the analysis unit 5, and the storage unit 6, and an operation unit 8 that can operate the control unit 7. The evaluator 1 includes only one measurement unit in FIG. 1, but may include a plurality of measurement units. In that case, a plurality of samples can be measured simultaneously.

The component in the culture supernatant may be any component as long as it enables the differentiation level to be evaluated, and an exosome, an exosome marker, and the like can be exemplified, but the component is not limited thereto. The exosome marker is not particularly limited, but may be selected from a group consisting of CD63, CD81, CD9, TSPAN9, HSP70, and HSP90.

The type of the cultured cells is not particularly limited, and pluripotent stem cells such as iPS cells and ES cells, stem cells such as mesenchymal stem cells, and other human-derived cells and animal-derived cells, and the like can be exemplified, and established cultured cells or primary cultured cells may be used.

A direction of differentiation is not particularly limited, and pluripotent stem cells include differentiation into all cell types, and neural cells such as nerve cells and glial cells, visceral cells such as hepatocyte cells and pancreatic cells, blood cells such as erythrocytes and leucocytes, muscle cells such as skeletal muscle and cardiac muscle, immune cells such as T cells, B cells, and dendritic cells, epithelial cells, mucosal cells, stromal cells, and the like can be exemplified. In particular, dopamine neural progenitor cells and dopaminergic neural cells are preferable.

The measurement unit includes an instrument capable of detecting exosomes or exosome markers and measuring a content of the markers, and may include, for example, a spectrofluorometer, an ELISA reader, a stereomicroscope, a fluorescence microscope, or the like.

The storage unit is not particularly limited, but a non-volatile storage is preferable, and a ROM, a flash memory, a magnetic storage (hard disk drive, floppy disk, magnetic tape, etc.), an optical disk, and the like can be exemplified. The storage unit 6 stores correlation information including a correlation between the content of the component and the differentiation level of the cultured cells. If the component is exosomes, it may be correlation information between a number of culture days for maximum differentiation and an exosome density in the culture supernatant, or may be correlation information between a signal intensity of differentiation markers and the exosome density in the culture supernatant. When the component is exosome markers, it may be correlation information between a signal intensity of the differentiation markers and a signal intensity when the exosome markers are detected. A format of the correlation information is not particularly limited, but the correlation information preferably indicates that the content of the component and the differentiation level of the cultured cells continuously correspond to each other. For example, a regression line or a regression curve indicating a relationship between the content of the component and the differentiation level of the cultured cells, or an equation representing any one of them, or the like can be exemplified. Note that in the present specification, the exosome density in the culture supernatant refers to a number of exosomes per unit volume of the culture supernatant.

The correlation between the content of the component and the differentiation level of the cultured cells can be found as follows.

When the component is exosomes, a correlation between a time after the differentiation induction of the cultured cells and the content of the exosomes in the culture supernatant is found. For example, it is conceivable that after the differentiation induction of the cultured cell, the culture supernatant is collected at predetermined time intervals, the content of the exosomes in the culture supernatant is measured, and the content of the exosomes is defined as 0% at the start of the differentiation induction, and is defined as 100% at a maximum differentiation time to create a regression line or a regression curve indicating the relationship between the content of the exosomes and the differentiation level. For example, in the case of dopamine neural progenitor cells, since it differentiates at the maximum on the 9th to 15th days, or the 10th to 14th days, or the 11th to 13th days, or the 12th day, 100% may be set on that day, 0% may be set on a differentiation induction start day, and the correlation between the content of the exosomes and the differentiation level (%) may be found. Alternatively, the correlation between the signal intensity of the differentiation markers and the content of the exosomes may be found at predetermined time intervals, a minimum value and a maximum value of the signal intensity of the differentiation markers may be defined as 0% and 100%, respectively, and the correlation of the content of the exosomes and the signal intensity (%) of the differentiation markers may be found. It is preferable to perform an experiment a plurality of times under the same differentiation condition, take an average, and create the regression line or the regression curve indicating the relationship between the content of exosomes and the differentiation level by a numerical value of the average. A method for measuring the content of exosomes is not particularly limited, but as an example in which the measurement can be easily performed while maintaining a closed space, an electrical resistance nanopulse method, a nanoparticle tracking method, a dynamic light scattering method, or a method using infrared spectroscopy or Raman spectroscopy can be exemplified.

When the component is exosome markers, the correlation between the signal intensity and the differentiation level when the exosome markers are detected is found. For example, it is conceivable that after the differentiation induction of the cultured cells, the culture supernatant is collected at predetermined time intervals, the markers in the culture supernatant are detected to measure the signal intensity, and the signal intensity is defined as 0% at the start of the differentiation induction, and is defined as 100% at a maximum differentiation time to create a regression line or a regression curve indicating the relationship between the signal intensity and the differentiation level. Again, it is preferable to perform the experiment a plurality of times under the same differentiation condition, take an average, and create a regression line or a regression curve indicating the relationship between the signal intensity and the differentiation level by a numerical value of the average. While a method for detecting the exosome markers and a method for measuring the content of the markers are not particularly limited, it is preferable in terms of simplicity that by using an antibody against the exosome markers and detecting fluorescence or enzyme bound to the antibody, the markers are detected to measure the content of the markers, and ELISA and the like can be exemplified.

Here, while a method for determining the differentiation level is not particularly limited, and a ratio of differentiated cells may be calculated by observation using a stereomicroscope of the measurement unit 4 or the like, it is preferable to determine the differentiation level by the markers of the differentiated cells.

### ==Automatic Cell Culture System==

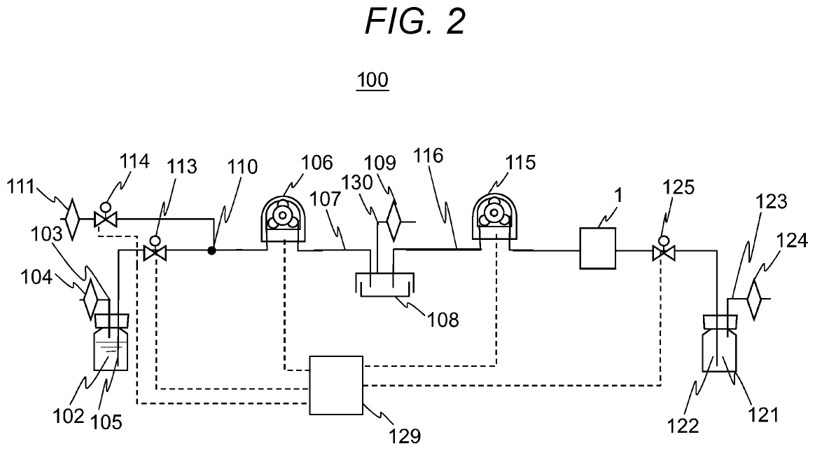

As shown in FIG. 2, the evaluator 1 may be connected to a culture device to configure an automatic cell culture system 100 as a whole. The culture device is a closed automatic culture device and is not particularly limited, and a device already developed may be applied, but one example is shown below.

The automatic cell culture system 100 of the present disclosure has a first container 102 for containing a first liquid and a second container 108 for containing the first liquid. The first container 102 is a container for storing a medium for cell culture, which is the first liquid. The second container 108 is a container for cell culture, and a shape thereof is not particularly limited, and for example, a dish, a bottle, or the like can be employed. The first container 102 and the second container 108 can be easily manufactured according to the technical common sense of those skilled in the art in consideration of the purpose. Each has an air pressure adjusting pipe 103 open to outside air, and has an end in a gas phase inside the container. The second container 108 can also be easily manufactured in consideration of the purpose by the technical common sense of those skilled in the art. The second container 108 has an air pressure adjusting pipe 130 open to the outside air, and has an end in a gas phase inside the container.

The culture device has a first liquid feeding pipe 105 for feeding the first liquid in the first container 102, and a second liquid feeding pipe 107 for feeding the first liquid in the first liquid feeding pipe 105 to the second container 108. The second liquid feeding pipe 107 has a first liquid feeding pump 106 to adjust liquid feeding into the second liquid feeding pipe 107. Each of the liquid feeding pipes can be easily manufactured according to the technical common sense of those skilled in the art. The first liquid feeding pipe 105 has a first valve 113 and a second valve 114, and opening and closing of each of the valves allows the presence or absence of liquid feeding to be switched.

Further, the culture device has a third container 121 for discarding the first liquid in the second container 108. The third container 121 can be easily manufactured according to the technical common sense of those skilled in the art in consideration of the purpose. The third container 121 has an air pressure adjusting pipe 123 open to the outside air, and has an end in a gas phase inside the container.

The culture device is further connected to a third liquid feeding pipe 116 for discharging the first liquid in the second container 108, and a fourth liquid feeding pipe 122 that is connected to the third liquid feeding pipe 116 to discharge the first liquid in the second container 108 into the third container 121 through the third liquid feeding pipe 116. The third liquid feeding pipe 116 for carrying the culture supernatant to the third container 121 has a second liquid feeding pump 115, and controls the liquid feeding in the third liquid feeding pipe 116. Each of the liquid feeding pipes can be easily manufactured according to the technical common sense of those skilled in the art.

The culture device further has the third liquid feeding pipe 116 connected from the second container 108 to the evaluator 1 and the fourth liquid feeding pipe 122 connected from the evaluator 1 to the third container 121. The liquid feeding pipe 122 has a third valve 125, and opening and closing allows the presence or absence of liquid feeding to be switched, so that the measurement of the evaluator 1 can be started/stopped. As described above, the liquid feeding pipe 122 has the third valve 125, and the opening and closing of the third valve 125 and ON and OFF of the evaluator 1 are adjusted in cooperation with each other.

The culture device may be provided with a control unit 129 exclusively, and it is preferable that activation of the pumps and the opening and closing of the valves, and the like can be automatically controlled.

### ==How to Activate the Automatic Cell Culture System==

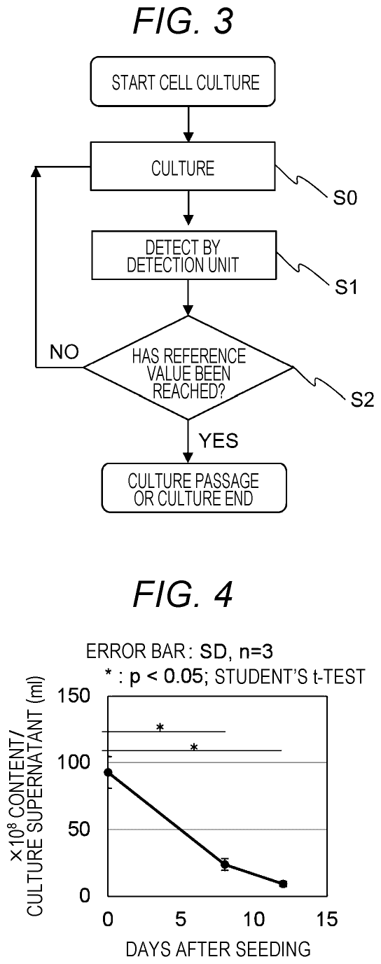

Hereinafter, a method for activating the automatic cell culture system 100 will be described in detail. Control of the automatic cell culture system 100 may be performed manually or by the control unit 7. FIG. 3 shows a flowchart when the control unit 7 is caused to perform the control.

First, the cell culture is started and the culture is continued (S0). After culturing in a predetermined period, the content of the exosomes or the exosome markers is measured by the measurement unit 4 for the exosomes in the culture supernatant, using the methods as described above (S1). The analysis unit 5 evaluates the differentiation level, using the relative information of the content of the exosomes or the exosome markers, and the differentiation level stored in the storage unit 6 in advance.

If the differentiation level does not reach a predetermined reference value (S2), data of the differentiation level calculated by the analysis unit of the evaluator is transmitted to the automatic cell culture device, for example, within 12 hours after the calculation, preferably within 6 hours, more preferably within 3 hours, still more preferably within 1 hour, and the cell culture is continued. If the differentiation level has reached the predetermined reference value (S2), the cells are subjected to passage as they are, or the data of the differentiation level is transmitted to the automatic culture device, and the cell culture ends.

### EXAMPLES

### Example 1

In the present example, it is shown that the exosome density in the culture supernatant decreases with time in accordance with a differentiation induction process of cells.

Specifically, differentiation induction was performed as follows (Doi, D. et al., Stem cell reports, 2.3, 337-350 (2014)). First, an iPS cell line 201B7 was used to seed 4×10⁵ cells per well in a 6-well dish coated with LM511-E8. When the cells reached a confluency after 4 days, a medium for growth (Stem Fit media) was replaced with a medium for differentiation (8% KSR, 0.1 mM MEM, NEAA (which are made by Invitrogen), sodium pyruvate (Sigma-Aldrich), 0.1 mM 2-mercaptoethanol-containing GMEM). In order to promote nerve differentiation, LDN193189 (STEMGENT) and A83-01 (Wako) were added, and in order to further perform differentiation induction of floor plate cells, on the 1st to 7th days after the medium replacement, purmorphamine and FGF8 (Wako) were added, and on the 3rd to the 12th days, CHIR99021 (Wako/STEMGENT) was added. In this way, differentiation induction into dopamine neural progenitor cells was performed for 12 days, and for the culture supernatants collected 0 days after the passage, 8 days after the passage, and 12 days after the passage, the exosome density was monitored by the electrical resistance nanopulse method.

FIG. 4 shows a relationship between a number of exosome granules per 1 mL of culture supernatant and a number of culture days after cell seeding. As is clear from the graph, the number of exosome granules in the culture supernatant decreased as a culture period increased.

Thus, as the iPS cell line differentiates into the dopamine neural progenitor cells, the number of exosome granules decreases.

### Example 2

In the present example, it is shown that a content of CD63, which is exosome markers in the culture supernatant, transiently increases in accordance with the differentiation induction process of cells, and when a differentiation induction level is low, the content of CD63 decreases in accordance with the differentiation induction level.

As in Example 1, the differentiation induction into the dopamine neural progenitor cells was performed for 12 days, and the content of CD63 in the culture supernatant was monitored daily by the ELISA method. As a culture condition for decreasing the differentiation induction level, culture was performed in a medium lacking FGF8, which is a differentiation inducer, and comparison was made with culture in a medium containing FGF8.

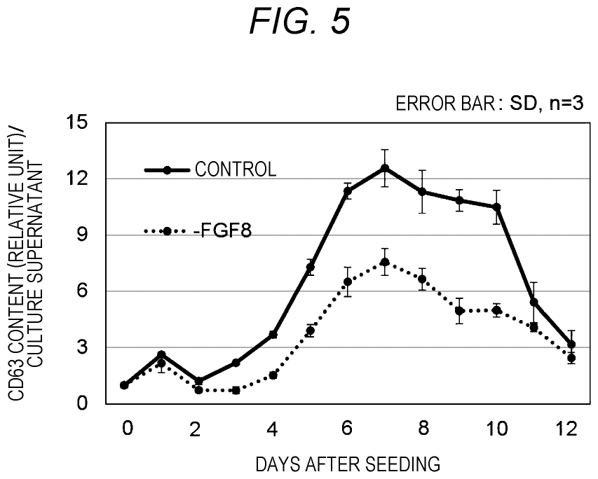

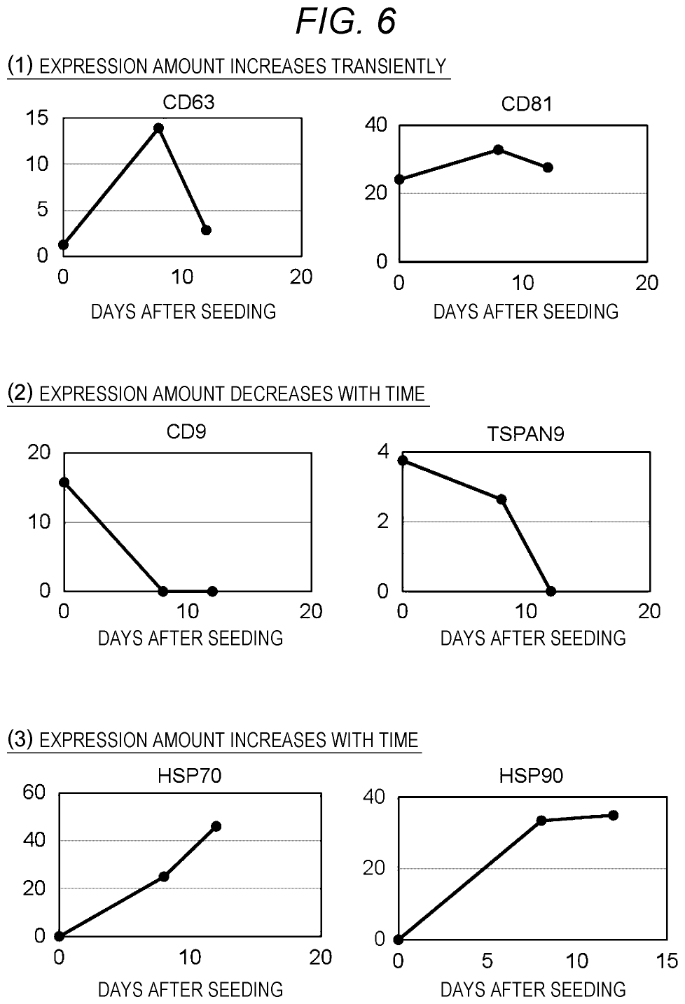

FIG. 5 shows a relationship between the content of CD63 in the total amount of the culture supernatant and the number of culture days after the cell seeding. The content of CD63 at the start of culture is set to 1, and the subsequent content of CD63 is shown as a relative value.

As is clear from the graph, a transient increase in the content was observed with the maximum on the 8th day during the 12-day culture. Under a condition lacking FGF8 (−FGF8), the content of CD63 decreased from the 3rd day on after the start of differentiation induction, as compared with a normal culture condition.

Thus, the content of CD63 in the culture supernatant can be a marker for the differentiation level.

### Example 3

In the present example, it is shown that the content of the exosome markers in the culture supernatant changes in three patterns in accordance with the differentiation induction process of cells. Specifically, an iPS cell line 201B7 was used to perform differentiation induction into dopamine neural progenitor cells for 12 days, and the contents of 6 kinds of proteins, which are exosome markers, in the culture supernatant were monitored. Note that as a condition for the differentiation induction into the dopamine neural progenitor cells, the same condition as that in Example 1 was applied.

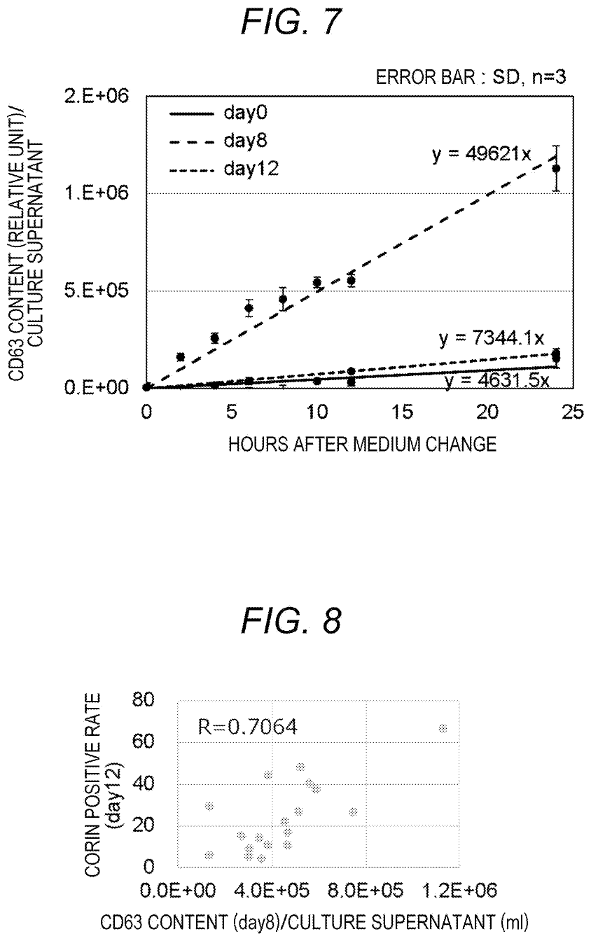

In the differentiation induction culture period from iPS cells to the dopamine neural progenitor cells, for the culture supernatants collected on the 0th day, the 8th day, and the 12th day after the start of differentiation induction, contents of CD63, CD81, CD, and TSPAN9, HSP70, and HSP90, which are exosome markers, were quantified by LC-MS. FIG. 7 shows a relationship between the content of each of the markers per culture supernatant and the number of culture days after cell seeding.

As is clear from the graph, changes of the contents of the markers are divided into three patterns: a group whose content increases transiently, a group whose content decreases successively, and a group whose content increases successively. Since the content changes in accordance with the change of the differentiation level, any of the groups can be used as markers of the differentiation level. In addition, the differentiation level can be evaluated with higher accuracy by combining results of evaluation of the differentiation level based on the plurality of markers of the differentiation level.

### Example 4

In the present example, it is shown that the content of CD63, which is an exosome marker, in the culture supernatant increases in proportion to an elapsed time after medium replacement. Specifically, an iPS cell line 201B7 was used to perform differentiation induction culture into dopamine neural progenitor cells for 12 days, and the content of CD63 after medium replacement was monitored. Note that as a condition for the differentiation induction into the dopamine neural progenitor cells, the same condition as that in Example 1 was applied.

The differentiation induction culture from the iPS cells into the dopamine neural progenitor cells was performed, and the medium was replaced on the 0th day, the 8th day, and the 12th day after the start of differentiation culture, and a medium replacement time was defined as 0 hours, and the culture supernatant was collected every 2 hours to quantify the content of CD63 in the culture supernatant by the ELISA method (PS Capture Exosome ELISA Kit, Fujifilm Wako Pure Chemical Industries, Ltd., quantification was performed according to the manufacturer's recommended protocol). FIG. 8 shows a relationship between the content of CD63 per amount of culture supernatant and the elapsed time after medium replacement.

As is clear from the graph, while the content of CD63 increased in proportion to the elapsed time after the medium replacement regardless of the number of elapsed days from the start of differentiation induction, the rate of increase varied, depending on the number of days after the start of differentiation culture. It is considered that in a stage where the content of the markers starts increasing at that point in Example 2 (results are shown in FIG. 5), an inclination of the straight line becomes large in the present example (results are shown in FIG. 7), and that in a stage where the content of the markers starts decreasing at that point in Example 2, the inclination of the straight line becomes small in the present invention example. Thus, by monitoring the content of the markers along with the elapsed time from the medium replacement, it is possible to evaluate the differentiation level, and further, to more accurately evaluate the differentiation level in combination with the results of Example 2.

### Example 5

In the present example, it is shown that the content of CD63 in the culture supernatant on the 8th day and a differentiation rate of the cultured cells on the 12th day correlate. Specifically, an iPS cell line 201B7 was used to perform differentiation induction into dopamine neural progenitor cells, the culture supernatant on the 8th day was collected, and the content of CD63 in the culture supernatant was quantified by the ELISA method. Furthermore, the cells on the 12th day were collected and a ratio of cells expressing CORIN, which is a marker for dopamine neural progenitor cells, was quantified by flow cytometry. This measurement was repeated 18 times, and results obtained with respect to the content of CD63 in the culture supernatant on the 8th day and a CORIN positive rate on the 12th day are shown in FIG. 8. Moreover, Pearson's correlation coefficient was calculated for these correlations. Note that as a condition for the differentiation induction into the dopamine neural progenitor cells, the same condition as that in Example 1 was applied.

As a result, Pearson's correlation coefficient R was 0.7064, and it was observed that there was a high correlation between the content of CD63 on the 8th day and an expression amount of the markers of dopamine neural progenitor cells on the 12th day.

Thus, the differentiation level during differentiation induction reflects the differentiation level in a final differentiation stage, and the differentiation rate of the final differentiated cells can be predicted by evaluating the differentiation level during differentiation induction. Since it is considered that the higher the differentiation rate of the final differentiated cells is, the higher cell quality finally obtained is, it can be said that evaluation of the differentiation level during differentiation induction is effective for predicting the quality of a final product.

What is claimed is:

1. An evaluator for evaluating a differentiation level of a cultured cell, comprising an analysis unit that evaluates the differentiation level of the cultured cell on the basis of a content of a component in a culture supernatant of the cultured cell.

2. The evaluator according to claim 1, further comprising a measurement unit that measures the content of the component in the culture supernatant of the cultured cell.

3. The evaluator according to claim 2, wherein the measurement unit includes a spectrofluorometer.

4. The evaluator according to claim 1, further comprising a storage unit that stores correlation information between the content of the component and the differentiation level of the cultured cell.

5. The evaluator according to claim 1, wherein the component is an exosome.

6. The evaluator according to claim 1, wherein the component is an exosome marker.

7. The evaluator according to claim 6, wherein the exosome marker is selected from a group consisting of CD63, CD81, CD9, TSPAN9, HSP70, and HSP90.

8. The evaluator according to claim 1, wherein the cell is a dopamine neural progenitor cell.

9. An automatic cell culture system, comprising:
a culture device including:
a cell culture container for culturing a cell; and
a drainage container for discarding a culture supernatant used for cell culture for a predetermined period; and
an evaluator including:
a measurement unit for measuring a content of a component in the culture supernatant; and
an analysis unit that evaluates a differentiation level of the cell from the content.

10. The automatic cell culture system according to claim 9,
wherein the culture device has a liquid feeding pipe for carrying the culture supernatant from the cell culture container to the drainage container through the evaluator,
the liquid feeding pipe has a valve that can be opened and closed, and
the opening and closing of the valve, and ON and OFF of the evaluator are adjusted in cooperation with each other.

11. The automatic cell culture system according to claim 9, wherein the evaluator further includes a determination unit that determines whether the cell culture is continued or terminated on a basis of the content.

12. The automatic cell culture system according to claim 9, wherein the component is an exosome.

13. The automatic cell culture system according to claim 9, wherein the component is an exosome marker.

14. The automatic cell culture system according to claim 13, wherein the exosome marker is selected from a group consisting of CD63, CD81, CD9, TSPAN9, HSP70, and HSP90.

15. An evaluation method for evaluating a differentiation level in a cultured cell, the evaluation method comprising evaluating a differentiation level of a cultured cell on the basis of a content of a component in a culture supernatant of the cultured cell.
